(19) United States
(12) Patent Application Publication
(10) Pub. No.: US 2022/0177891 A1
(43) Pub. Date: Jun. 9, 2022

(54) HIPK INHIBITORS AND METHODS OF USE THEREOF

(71) Applicants: Board of Regents, The University of Texas System, Austin, TX (US); Vibliome Therapeutics, LLC, New York, NY (US)

(72) Inventors: Qiang SHEN, Houston, TX (US); Gary A. FLYNN, New York, NY (US)

(73) Assignees: Board of Regents, The University of Texas System, Austin, TX (US); Vibliome Therapeutics, LLC, New York, NY (US)

(21) Appl. No.: 17/432,039

(22) PCT Filed: Feb. 19, 2020
(86) PCT No.: PCT/US2020/018850

§ 371 Date: Aug. 18, 2021

Related U.S. Application Data
(60) Provisional application No. 62/807,594, filed on Feb. 19, 2019.

(51) Int. Cl. C12N 15/113; A61P 35/04; A61K 31/4439; A61K 51/04; A61K 45/06; A61K 31/713; A61K 31/422

(52) U.S. Cl. CPC C12N 15/1137; A61P 35/04; A61K 31/4439; A61K 51/0455; C12N 2320/31; A61K 31/713; A61K 31/422; C12N 2310/14; A61K 45/06

(57) ABSTRACT

Method of treating cancer, such as a metastatic cancer, with inhibitors of homeodomain interacting protein kinase 4 (HIPK4) are provided. Related therapeutic compositions are also disclosed.

This application claims the benefit of U.S. Provisional Patent Application No. 62/807,594, filed Feb. 19, 2019, the entirety of which is incorporated herein by reference.

### BACKGROUND OF THE INVENTION

### 1. Field of the Invention

The present invention relates generally to the field of cancer biology. More particularly, it concerns compositions and methods for the detection and inhibition of HIPK4 in metastatic cancers.

### 2. Description of Related Art

Metastasis occurs in approximately 40% of epithelial cancer patients and accounts for 90% of cancer-related deaths. As the 2^{nd} leading cause of death in women, breast cancer is a global health threat to middle-aged women. Patients with localized breast cancer are predicted to achieve a 5-year survival in approximate 99% of patients, however, only about 27% of breast cancer patients with distant metastasis achieve 5-year survival. Invasive progression of breast cancer drives the development of metastatic lesions in major organs including lung, brain, bone, or liver, leading to approximately 40,300 deaths per year in the United States alone. These facts manifest the urgent needs of identifying key driving determinants in breast cancer metastatic progression, as well as the need to discover effective strategies to target those metastasis determinants.

Because activated protein kinases are critically involved in cancer growth and progression, kinase inhibitors have played an increasingly prominent role in cancer therapy. However, there is a lack of known metastasis-specific kinases and corresponding targeted therapy against metastatic breast cancer. Therefore, identifying metastasis-specific kinases and their inhibitors could provide new biomarkers and treatments for metastatic cancers.

Homeodomain Interacting Protein Kinase 4 (HIPK4) belongs to a new family of enzymes which interact in a yeast two-hybrid screen with domains of other proteins. These proteins comprise a conserved protein kinase domain and a domain which interacts with other proteins, and therefore the protein kinases are thus referred to as homeodomain-interacting protein kinases (HIPKs). To date, four HIPK isoforms are known, designated HIPK1-HIPK4. (Choi et al., 1998). HIPK1-HIPK3 have been detected in the nucleus, and several substrates have been identified. The sequence of HIPK4 was claimed in WO2004087901A2, though little else is known about HIPK4.

Functional information relating to human HIPKs has been limited (Kim et al., 1998; Moilanen et al., 1998; Rochat-Steiner et al., 2000; Ecsedy et al., 2003; Zhang et al., 2005; Shikegi et al., 2007). HIPKs form a family of highly conserved kinases that are involved in diverse cellular functions including regulation of cell death, survival, proliferation, and differentiation (Rinaldo et al., 2008). Additionally, HIPKs have been implicated in modulating the cellular response to DNA damage, such as genotoxic stress. HIPK4 was also identified as a marker of cell death and survival (He et al., 2010).

The recent development of multiple kinase inhibitors has provided another avenue by which to treat cancers and immunosuppressive cell populations. The type-II kinase inhibitor Sorafenib is approved for the treatment of several human cancers is an inhibitor of several human cancers, and is believed to inhibit tumor growth via anti-angiogenesis, cell cycle arrest, and induction of apoptosis, has been shown to modulate immunsuppressive cell populations in a murine liver cancer model. (Cao et al, 2011). While Sorafenib is capable of inhibiting the 4 known HIPKs and HIPK4 potently (IC₅₀=3.3 nM), it lacks selectivity (Davis et al., 2011). Therefore, specific and potent inhibitors of HIPKs could be of potential therapeutic interest.

### SUMMARY OF THE INVENTION

In some embodiments, the present disclosure provides methods of treating cancer in a subject comprising administering an effective amount of an inhibitor of homeodomain interacting protein kinase 4 (HIPK4) to the subject. In some aspects, the methods are further defined as methods for preventing and inhibiting cancer metastasis. In some aspects, the methods are further defined as methods for inhibiting cancer cell migration or invasion. In some aspects, the cancer is an invasive or progressive cancer. In some aspects, the cancer is oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer. In some aspects, the cancer is a breast cancer. In further aspects, the cancer is an invasive breast cancer with or without metastatic diseases or lesions.

In some aspects, the subject has a metastatic cancer. In some aspects, the subject has a metastasis developed in the lungs, brain, bone, or liver. In some aspects, the subject has a metastasis in multiple organs. In some aspects, the inhibitor of HIPK4 is an inhibitory nucleic acid molecule, such as a siRNA, shRNA, miRNA, dsRNA, a ribozyme or antisense nucleic acid. In some aspects, the inhibitor of HIPK4 is a small molecule kinase inhibitor. In further aspects, the small molecule inhibitor is VIB-MDA-001, VIB-MDA-002, VIB-MDA-003, VIB-MDA-004, VIB-MDA-005, VIB-MDA-006, VIB-MDA-007, VIB-MDA-008, or VIB-MDA-009. In some aspects, the small molecule inhibitor has the general structure:

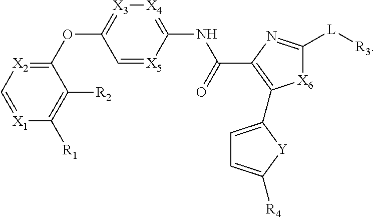

In some aspects, the small molecule inhibitor is VIB-MDA-003. In some aspects, the small molecule inhibitor has the structure:

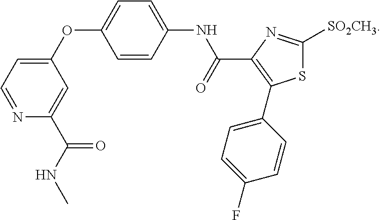 [SMILES: CNC(=O)c1cc(Oc2ccc(NC(=O)c3nc(S(C)(=O)=O)sc3-c3ccc(F)cc3)cc2)ccn1]

In some aspects, the small molecule inhibitor is VIB-MDA-001. In some aspects, the small molecule inhibitor has the structure:

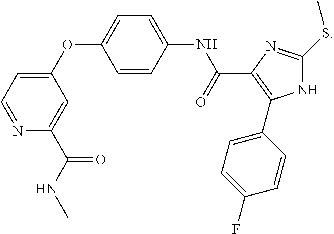 [SMILES: CNC(=O)c1cc(Oc2ccc(NC(=O)c3nc(SC)[nH]c3-c3ccc(F)cc3)cc2)ccn1]

In some aspects, the small molecule inhibitor is VIB-MDA-002. In some aspects, the small molecule inhibitor has the structure:

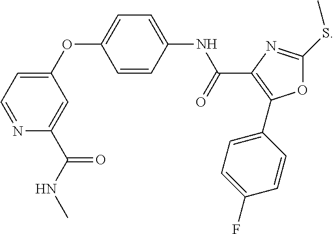 [SMILES: CNC(=O)c1cc(Oc2ccc(NC(=O)c3nc(SC)oc3-c3ccc(F)cc3)cc2)ccn1]

In further aspects, the small molecule inhibitor comprises one of the inhibitors disclosed in U.S. Pat. Nos. 9,221,805; 9,833,455 or International PCT Publication WO2013022766, each of which is incorporated herein by reference.

In some aspects, the subject has been determined to have a cancer with an elevated level of HIPK4 expression. In some aspects, the inhibitor of HIPK4 is administered more than once. In further aspects, the inhibitor of HIPK4 is administered 1, 2, 3, 4, 5, 6, or more times per week. In some aspects, the inhibitor of HIPK4 is administered daily. In some aspects, the inhibitor of HIPK4 is administered on a continuous basis. In some aspects, the methods further comprise administering an additional anti-cancer therapy such as chemotherapy, radiotherapy, gene therapy, surgery, hormonal therapy, anti-angiogenic therapy or immunotherapy. In some aspects, the inhibitor of HIPK4 is administered intravenously, subcutaneously, intraosseously, orally, transdermally, via inhalation, in sustained release, in controlled release, in delayed release, as a suppository, or sublingually. In some aspects, administering the inhibitor of HIPK4 comprises local, regional or systemic administration. In some aspects, the subject is a human.

In other embodiments, the present disclosure provides methods of treating a subject having a cancer comprising:

(a) obtaining a sample from the subject;

(b) determining the level of homeodomain interacting protein kinase 4 (HIPK4) expression or kinase function in the sample; and

(c) administering an effective amount of an inhibitor of HIPK4 to a subject determined to have an elevated level of HIPK4 expression or kinase function.

In still other embodiments, the present disclosure provides methods of predicting a response to an inhibitor of HIPK4 in a subject having a cancer comprising detecting the level of HIPK4 expression or kinase function in a tissue sample obtained from the subject, wherein if the sample exhibits increased expression or kinase activity of HIPK4, then the subject is predicted to have a favorable response to a HIPK4 inhibitor therapy. In some aspects, the level of HIPK4 expression is the level of HIPK4 mRNA expression. In some aspects, the level of HIPK4 expression is the level of HIPK4 protein expression. In some aspects, the level of HIPK4 activity is the level of HIPK4 kinase function. In some aspects, the sample is from saliva, blood, urine, or tumor tissue. In some aspects, the level of HIPK4 expression is determined by PCR analyses. In some aspects, the level of HIPK4 function is determined by kinase analyses.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Migration and Invasion of GI101 and its derivative lines. GILM2 and GILM3 shown enhanced migration and invasion compared to GI101A. *: p<0.05.

FIG. 2: Overlap of Activated Kinases in GL101, GILM2, and GILM3 cell lines. Comparisons of GI101A to GILM2/GILM3 were made from the expression data of the kinase arrays.

FIGS. 3A-3F: HIPK4 expression is correlated with breast cancer progression, metastasis, and prognosis. HIPK4 mRNA levels were analyzed in cancer tissue datasets. Datasets are as follows: (A) Finak Breast data set, (B) TCGA Breast Dataset, (C) Ma Breast 4 Dataset, (D) Kao Breast Dataset, (E) Loi Breast Dataset, and (F) Kao Breast Dataset. HIPK4 upregulation correlates with invasive breast cancer (A), stage II and II breast cancers (B), local lymph node metastasis status (C), distant metastatic events (D), local recurrences at 3 years (E), and 5-year prognosis (F).

FIGS. 4A-4E: HIPK4 protein expression in primary and metastatic breast tissues. (A) Normal breast tissue, (B) Invasive breast cancer, (C) Recurrent breast cancer, (D) Lymph node metastasis, and (E) Normal lymph node control were stained with anti-HIPK4 antibody at a dilution of 1:50.

FIG. 5: Discovery of specific HIPK4 inhibitors. The identified inhibitors show high specificity for HIPK4.

FIGS. 6A-6C: HIPK4 inhibitor VIB-MDA-002 activity. VIB-MDA-002 shows high efficacy in blocking HIPK4 kinase activity. (A) VIB-MDA-002 demonstrates selectivity for HIPK4 in comparison with Sorafenib. (B) IC50 and kinase activity of HIPK4 and DDR2 following treatment with VIB-MDA-002. (C) Structures of Sorafenib and VIB-MDA-002.

FIGS. 7A-7D: Gain or loss of HIPK4 significantly affects migration and invasion. (A)&(B): si-HIPK4 in GILM2/MDA-MB-231 cells. (C)&(D): HIPK4-overexpression in GI101A/MDA-MB-231 cells. All transient transfection assays. *: p<0.05.

FIG. 8: Confirmation of HIPK4 DNA loci edited by Cas9 in MDA-MB-231 cells, T7EN1 assay.

FIG. 9: HIPK4 inhibitors show high selectivity in inhibiting HIPK4 protein. Protein levels of HIPK4, FAK, and p-FAK in the presence of the VIB-MDA compounds. HIPK1-3 protein levels were not significantly altered by VIB-MDA compounds (data not shown).

FIGS. 10A-10D: HIPK4 inhibitors significantly inhibit cellular migration and invasion of metastatic EPBC cell line GILM3. (A-B) Migration of GILM3 cells and their quantification in the presence of VIB-MDA compounds. (C-D) Second data set for invasion of GILM3 cells and their quantification in the presence of VIB-MDA compounds.

FIGS. 11A & 11B: HIPK4 overexpression significantly enhances cellular motility and HIPK4 inhibitors significantly inhibit HIPK4-induced cellular migration and HIPK4 protein levels. (A) Migration of cells overexpressing HIPK4 and treated with VIB-MDA compounds. (B) Western blots cells overexpressing HIPK4 and treated with VIB-MDA compounds.

FIG. 12: Variation of Aryl Scaffold in Small Molecule HIPK4 Inhibitors Modulates Potency. Changing the substitution and/or ring structure of the aryl scaffold of VIB-MDA compounds leads to modulation of potency.

### DESCRIPTION OF ILLUSTRATIVE EMBODIMENTS

The present application relates to the inhibition of homeodomain-interacting protein kinase 4 (HIPK4) for the treatment of cancer. Provided herein are methods and compositions for the inhibition of HIPK4, and use of these methods and compositions for the prevention and treatment of cancers, particularly metastatic cancers.

### I. HOMEODOMAIN-INTERACTING PROTEIN KINASES

Homeodomain-interacting protein kinases (Hipks) are evolutionarily conserved, and vertebrates possess four Hipks, Hipk1-Hipk4, whereas Drosophila and Caenorhabditis elegans have only one Hipk each. The HipK family of proteins was first identified in a yeast two-hybrid screen to identify cofactors for the NKx-1.2 family of homeoproteins (Kim et al., 1998). Hipk1 and Hipk2 were first identified, and have greater than 93% amino acid identity in their kinaser domains. Hipk3 was identified later, and is slightly less conserved, sharing only about 87% identity with Hipk1 and Hipk2 (Van der Laden et al., 2015). Hipk4 is the most divergent member of the family and shares only about 50% homology within the kinase domain (Arai et al., 2007; He et al., 2010). Hipk family members are expressed in dynamic temporal and spatial patterns, highlighting their important roles during development (reviewed by Blaquiere and Verheyen, 2017). Hipk protein levels are highly regulated by post-translational modification and proteasomal degradation (Saul and Schmitz, 2013). Hipk family members are reported to have distinct and contradictory effects on cell proliferation and tissue growth. Overexpression of Drosophila Hipk causes tissue overgrowths in the wing, eye and legs in a dose dependent manner (Chen and Verheyen, 2012; Lee et al., 2009a; Poon et al., 2012). In C. elegans, Hpk-1 promotes proliferation of the germline cells, and loss of hpk-1 reduces the number of proliferating cells and size of the mitotic region (Berber et al., 2013). Hipk2−/− mice have growth deficiencies, and 40% die prematurely (Chalazonitis et al., 2011; Sjolund et al., 2014; Trapasso et al., 2009). In normal human skin, Hipk2 protein expression is enriched in basal proliferating cells, whereas it is undetectable in nonproliferating cells (Iacovelli et al., 2009), and expression is reactivated when cells are stimulated to proliferate, suggesting a close link between Hipk protein function and cell proliferation. Mouse embryo fibroblasts (MEFs) from Hipk2−/− knockout mice show reduced proliferation (Trapasso et al., 2009), whereas another study claimed that such cells proliferated more than wild type (Wei et al., 2007). From these studies, it is clear that much remains to be learned about the roles of Hipk family protein kinases in proliferation and cell behavior.

Hipks regulate numerous signaling pathways required for the development of healthy tissues (reviewed by Blaquiere and Verheyen, 2017). Both Drosophila and vertebrate Hipks can modulate Wnt signaling in many ways (Hikasa and Sokol, 2011: Hikasa et al., 2010; Kuwahara et al., 2014; Lee et al., 2009b; Louie et al., 2009; Shimizu et al., 2014; Swarup and Verheyen, 2011; Wu et al., 2012). Hipk2 is the best-characterized vertebrate Hipk family member. Studies in cell culture and cancer samples reveal conflicting results (Blaquiere and Verheyen, 2017). For example, Hipk2 acts as a tumor suppressor in the context of p53-mediated cell death after lethal DNA damage (Hofmann et al., 2003), and reduced expression of Hipk proteins is seen in several cancer types (Lavra et al., 2011; Pierantoni et al., 2002; Ricci et al., 2013; Tan et al., 2014). By contrast, Hipk2 is elevated in certain cancers, including cervical cancers, pilocytic astrocytomas and colorectal cancer cells, and in other diseases, such as thyroid follicular hyperplasia (Al-Beiti and Lu, 2008; Cheng et al., 2012; D'Orazi et al., 2006; Deshmukh et al., 2008; Jacob et al., 2009; Lavra et al., 2011; Saul and Schmitz, 2013; Yu et al., 2009). Human Hipk1 is also found at elevated levels in certain cancer cell lines and tissue samples (Kondo et al., 2003; Rey et al., 2013). Hipk4 can phosphorylate p53Ser9, which enhances p53-mediated transcriptional repression (Arai et al., 2007).

Numerous generic protein kinase inhibitors can influence Hipk family members, though very few have been described to be specific to Hipks. D-115893 causes Hipk delocalization and distribution throughout the cell, in addition to blocking p53Ser46 phosphorylation (de la Vega et al., 2011). A specific Hipk2 inhibitor named TBID was identified that can block phosphorylation of both p53 and a generic substrate by endogenous Hipk2 in human T-lymphoblastoid cells (Cozza et al., 2014). TBID is also capable of inhibiting Hipk1 and Hipk3 at lower efficiencies.

Selective HIPK4 inhibitors may also be used as recognition elements for targeted protein degradation therapies, such as proteolysis targeting chimera (PROTAC) degradation (Neklesa et al., 2017). Targeted protein degradation using the PROTAC technology is a therapeutic method to address diseases driven by the aberrant expression of a disease-causing protein. PROTAC molecules are bifunctional small molecules that simultaneously bind a target protein and an E3-ubiquitin ligase, thus causing ubiquitination and degradation of the target protein by the proteasome. Like small molecules, PROTAC molecules possess good tissue distribution and the ability to target intracellular proteins. PROTACs can degrade proteins regardless of their function. This includes the currently “undruggable” proteome, which comprises approximately 85% of all human proteins. Other beneficial aspects of protein degradation include the ability to target overexpressed and mutated proteins, as well as the potential to demonstrate prolonged pharmacodynamics effect beyond drug exposure. Lastly, due to their catalytic nature and the pre-requisite ubiquitination step, exquisitely potent molecules with a high degree of degradation selectivity for HIPK4 can be designed.

The integrated use of PET imaging probes with very similar properties and inhibition profiles as the therapeutic agent of interest will offer significant and unique advantages for selection of most appropriate patient populations for clinical studies. Targeted monitoring of kinases using PET imaging represents a significant step toward the realization of personalized medicine. Small molecule inhibitors such as those described in U.S. Pat. Nos. 9,221,805, 9,833,455, or PCT Publication No. WO 2013/022766 may be used in combination with PET imaging agents or may themselves act as PET imaging agents. These compounds may also be modified to incorporate a moiety capable of being detected by PET imaging, such as [18]F. For example, introduction of [18]F at the 4-fluorophenyl azole substituent of VIB-MDA-001, VIB-MDA-002, VIB-MDA-003, VIB-MDA-004, VIB-MDA-005, VIB-MDA-006, VIB-MDA-007, VIB-MDA-008, VIB-MDA-009 can be achieved by replacement of a 4-hydroxyl group in the corresponding precursor compounds using PhenoFluor™ (1,3-Bis(2,6-diisopropylphenyl)-2,2-difluoro-2,3-dihydro-1H-imidazole; Scheme 1) or other methods for deoxyfluorination (see for example Ritter et al., 2017).

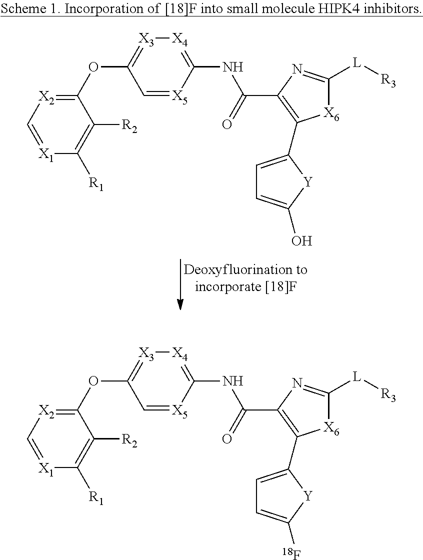

### II. PHARMACEUTICAL FORMULATIONS AND TREATMENT OF CANCER

A. Cancers

Cancer results from the outgrowth of a clonal population of cells from tissue. The development of cancer, referred to as carcinogenesis, can be modeled and characterized in a number of ways. An association between the development of cancer and inflammation has long-been appreciated. The inflammatory response is involved in the host defense against microbial infection, and also drives tissue repair and regeneration. Considerable evidence points to a connection between inflammation and a risk of developing cancer, i.e., chronic inflammation can lead to dysplasia.

Cancer cells to which the methods of the present disclosure can be applied include generally any cell that expresses HIPK4, and more particularly, that overexpresses HIPK4. An appropriate cancer cell can be a breast cancer, lung cancer, colon cancer, pancreatic cancer, renal cancer, stomach cancer, liver cancer, bone cancer, hematological cancer (e.g., leukemia or lymphoma), neural tissue cancer, melanoma, ovarian cancer, testicular cancer, prostate cancer, cervical cancer, vaginal cancer, or bladder cancer cell. In addition, the methods of the disclosure can be applied to a wide range of species, e.g., humans, non-human primates (e.g., monkeys, baboons, or chimpanzees), horses, cattle, pigs, sheep, goats, dogs, cats, rabbits, guinea pigs, gerbils, hamsters, rats, and mice. Cancers may also be recurrent, metastatic and/or multi-drug resistant, and the methods of the present disclosure may be particularly applied to such cancers so as to render them resectable, to prolong or re-induce remission, to inhibit angiogenesis, to prevent or limit metastasis, and/or to treat multi-drug resistant cancers. At a cellular level, this may translate into killing cancer cells, inhibiting cancer cell growth, or otherwise reversing or reducing the malignant phenotype of tumor cells.

B. Formulation and Administration

The present disclosure provides pharmaceutical compositions comprising HIPK4 inhibitors. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Other suitable pharmaceutical excipients include starch, glucose, lactose, sucrose, saline, dextrose, gelatin, malt, rice, flour, chalk, silica gel, sodium stearate, glycerol monostearate, talc, sodium chloride, dried skim milk, glycerol, propylene glycol, water, ethanol and the like.

The compositions can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The HIPK4 inhibitors of the present disclosure may include classic pharmaceutical preparations. Administration of these compositions according to the present disclosure will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions, described supra. Of particular interest is direct intratumoral administration, perfusion of a tumor, or administration local or regional to a tumor, for example, in the local or regional vasculature or lymphatic system, or in a resected tumor bed.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

C. Combination Therapies

In the context of the present disclosure, it also is contemplated that the HIPK4 inhibitors described herein could be used similarly in conjunction with chemo- or radiotherapeutic intervention, or other treatments. It also may prove effective, in particular, to combine HIPK4 inhibitors with other therapies that target different aspects of kinase expression or function.

To kill cells, inhibit cell growth, inhibit metastasis, inhibit angiogenesis or otherwise reverse or reduce the malignant phenotype of tumor cells, using the methods and compositions of the present disclosure, one would generally contact a “target” cell with a HIPK4 inhibitor according to the present disclosure and at least one other agent. These compositions would be provided in a combined amount effective to kill or inhibit proliferation of the cell. This process may involve contacting the cells with the HIPK4 inhibitor according to the present disclosure and the other agent(s) or factor(s) at the same time. This may be achieved by contacting the cell with a single composition or pharmacological formulation that includes both agents, or by contacting the cell with two distinct compositions or formulations, at the same time, wherein one composition includes the HIPK4 inhibitor according to the present disclosure and the other includes the other agent.

Alternatively, the HIPK4 inhibitor therapy may precede or follow the other agent treatment by intervals ranging from minutes to weeks. In embodiments where the other agent and the HIPK4 inhibitor are applied separately to the cell, one would generally ensure that a significant period of time did not expire between each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the HIPK4 inhibitor or the other agent will be desired. Various combinations may be employed, where a HIPK4 inhibitor according to the present disclosure therapy is “A” and the other therapy is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A _A/A/B/A A/B/B/B B/A/B/B B/B/A/B

Administration of the therapeutic agents of the present invention to a patient will follow general protocols for the administration of that particular secondary therapy, taking into account the toxicity, if any, of the antibody treatment. It is expected that the treatment cycles would be repeated as necessary. It also is contemplated that various standard therapies, as well as surgical intervention, may be applied in combination with the described cancer therapies.

The skilled artisan is directed to “Remington's Pharmaceutical Sciences” 15th Edition, Chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### 1. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy. The present invention contemplates any chemotherapeutic agent that may be employed or known in the art for treating or preventing cancers.

### 2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic agent and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

### 3. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T-cells and NK cells. The combination of therapeutic modalities, i.e., direct cytotoxic activity and inhibition or reduction of Fortilin would provide therapeutic benefit in the treatment of cancer.

Immunotherapy could also be used as part of a combined therapy. The general approach for combined therapy is discussed below. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as mda-7 has been shown to enhance anti-tumor effects (Ju et al., 2000).

As discussed earlier, examples of immunotherapies currently under investigation or in use are immune adjuvants (e.g., Mycobacterium bovis, Plasmodiur falciparum, dinitrochlorobenzene and aromatic compounds) (U.S. Pat. Nos. 5,801,005; 5,739,169; Hui and Hashimoto, 1998; Christodoulides et al., 1998), cytokine therapy (e.g., interferons, and; IL-1, GM-CSF and TNF) (Bukowski et al., 1998: Davidson et al., 1998; Hellstrand et al., 1998) gene therapy (e.g., TNF, IL-1, IL-2, p53) (Qin et al., 1998; Austin-Ward and Villaseca, 1998; U.S. Pat. Nos. 5,830,880 and 5,846,945) and monoclonal antibodies (e.g., anti-ganglioside GM2, anti-HER-2, anti-p185) (Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Combination therapy of cancer with herceptin and chemotherapy has been shown to be more effective than the individual therapies. Thus, it is contemplated that one or more anti-cancer therapies may be employed with the tumor-associated HLA-restricted peptide therapies described herein.

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated antigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders was few compared to those who did not respond.

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Human monoclonal antibodies are employed in passive immunotherapy, as they produce few or no side effects in the patient. However, their application is somewhat limited by their scarcity and have so far only been administered intralesionally. Human monoclonal antibodies to ganglioside antigens have been administered intralesionally to patients suffering from cutaneous recurrent melanoma (Irie & Morton, 1986). Regression was observed in six out of ten patients, following, daily or weekly, intralesional injections. In another study, moderate success was achieved from intralesional injections of two human monoclonal antibodies (Irie et al., 1989). Possible therapeutic antibodies include, but are not limited to, anti-TNF, anti-CD25, anti-CD3, anti-CD20, CTLA-4-IG, anti-CTLA4, anti-CD28, anti-PD-1, PD-L1, anti-PD-2, and PD-L2.

It may be favorable to administer more than one monoclonal antibody directed against two different antigens or even antibodies with multiple antigen specificity. Treatment protocols also may include administration of lymphokines or other immune enhancers as described by Bajorin et al. (1988). The development of human monoclonal antibodies is described in further detail elsewhere in the specification.

### 4. Gene Therapy

In yet another embodiment, the secondary treatment is a gene therapy in which a therapeutic polynucleotide is administered before, after, or at the same time as the tumor-associated HLA-restricted peptide is administered. Delivery of a vector encoding the tumor-associated HLA-restricted peptide in conjunction with a second vector encoding one of the following gene products will have a combined anti-hyperproliferative effect on target tissues. Alternatively, a single vector encoding both genes may be used. A variety of proteins are encompassed within the invention, some of which are described below. Various genes that may be targeted for gene therapy of some form in combination with the present invention are well known to one of ordinary skill in the art and may comprise any gene involved in cancers.

Inducers of Cellular Proliferation. The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity. The proteins Jun, Fos and Myc are proteins that directly exert their effects on nuclear functions as transcription factors.

Inhibitors of Cellular Proliferation. The tumor suppressor oncogenes function to inhibit excessive cellular proliferation. The inactivation of these genes destroys their inhibitory activity, resulting in unregulated proliferation. The most common tumor suppressors are Rb, p53, p21 and p16. Other genes that may be employed according to the present invention include APC, DCC, NF-1, NF-2, WT-1, MEN-I, MEN-II, zac1, p73, VHL, C-CAM, MMAC1/PTEN, DBCCR-1, FCC, rsk-3, p27, p27/p16 fusions, and p21/p27 fusions.

Regulators of Programmed Cell Death. Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985: Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins that share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

### 5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 6. Chemical Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means=NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means=S; “sulfonyl” means —S(O)₂—; and “sulfinyl” means —S(O)—.

In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, isotopes of carbon include ¹³C and ¹⁴C, and isotopes of fluorine include ¹⁸F.

In the context of chemical formulas, the symbol “-” means a single bond, “=” means a double bond, and “=” means triple bond. The symbol “----” represents an optional bond, which if present is either single or double. The symbol ‘ [symbol] ’ represents a single bond or a double bond. Thus, the formula

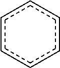

covers, for example

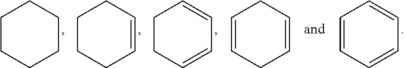

And it is understood that no one such ring atom forms part of more than one double bond. Furthermore, it is noted that the covalent bond symbol “-”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

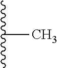

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol ‘ [symbol] ’ means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

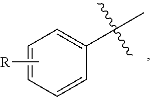

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

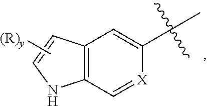

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “cycloalkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. Except as noted below, every carbon atom is counted to determine whether the group or compound falls with the specified number of carbon atoms. For example, the group dihexylamino is an example of a dialkylamino_{(C=12)} group; however, it is not an example of a dialkylamino_{(C=6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n +2 electrons in a fully conjugated cyclic π system. An aromatic compound or chemical group may be depicted as a single resonance structure; however, depiction of one resonance structure is taken to also refer to any other resonance structure. For example:

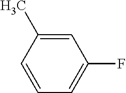

is also taken to refer to

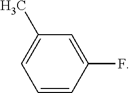

Aromatic compounds may also be depicted using a circle to represent the delocalized nature of the electrons in the fully conjugated cyclic π system, two non-limiting examples of which are shown below:

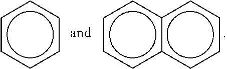

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂(i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃(tert-butyl, t-butyl, t-Bu or ^{t}Bu), and —CH₂C(CH₃)₃(neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CHCH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “cycloalkenyl” refers to a monovalent non-aromatic group with a carbon atom as the point of attachment, said carbon atom forming part of one or more non-aromatic ring structures, each with three to eight ring atoms, wherein the cycloalkenyl group comprises at least one non-aromatic carbon-carbon double bond, no carbon-carbon triple bonds, and no atoms other than carbon and hydrogen. Non-limiting examples include: cyclopropenyl, cyclobutenyl, cyclopentenyl, or cyclohexenyl. As used herein, the term does not preclude the presence of one or more alkyl groups (carbon number limitation permitting) attached to a carbon atom of the non-aromatic ring structure. A “cycloalkene” refers to the class of compounds having the formula H—R, wherein R is cycloalkyl as this term is defined above.

The term “heterocycloalkenyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkenyl group comprises at least one nonaromatic carbon-carbon double bond, no carbon-carbon triple bonds, and no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. The term “N-heterocycloalkenyl” refers to a heterocycloalkenyl group with a nitrogen atom as the point of attachment. A “heterocycloalkene” refers to the class of compounds having the formula H—R, wherein R is heterocycloalkenyl. 3,4-Dihydro-2H-pyran and 2,3-dihydropyrrole are non-limiting examples of heterocycloalkenes.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

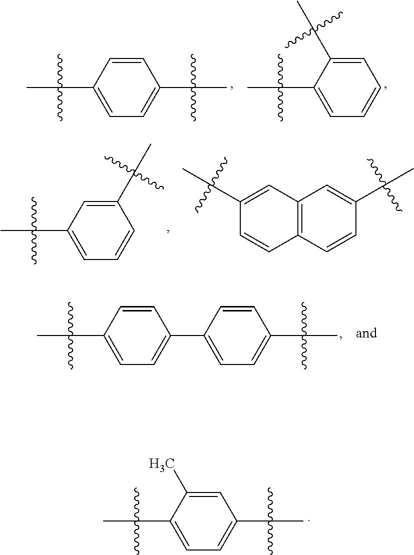

An “arene” refers to the class of compounds having the formula H—R, wherein R is aryl as that term is defined above. Benzene and toluene are non-limiting examples of arenes.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl (Im), isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

### Kinase Array

The antibodies array was performed following manufacture's guide. Briefly, GI101A, GILM2, GILM3 cells in the logarithmic phase were seeded in 10 cm cell culture dishes and incubated for 24 h. After that cells were harvest and lysed with the extraction buffer, 100 μg of cell lysate in 50 μL of reaction mixture were labeled with biotin in 10 N, N-dimethyformamide. The resulting biotin-labeled proteins were diluted 1:20 in coupling solution before applying to the array for conjugation. To prepare the antibody microarray, it was first blocked with blocking solution for 45 min at room temperature, rinsed with Milli-Q grade water. Finally, the array was incubated with the biotinlabeled cell lysates at room temperature for 2 h. After the array slide was washed with 1×wash solution, the conjugated-labeled protein was detected using Cy3-streptavidin. The slides were scanned and the signals were analyzed by Full Moon BioSystems, Inc. The data was analyzed using GraphPad software.

### Cell Migration and Invasion Assay

The in vitro migration and invasion assay was carried out using transwell chamber. Briefly, 0.2-2.0×10⁵ cells/ml cells were suspended in serum-free medium with 5% bovine serum albumin and then were placed in the upper compartment, and medium with 5% fetal bovine serum was added in the lower compartment. Following incubation in cell culture incubator for 6-32 h, non-migratory and noninvasive cells on the upper surface were gently removed by a cotton swab. The cells on the bottom of membrane were stained with cell stain solution at room temperature for 10 min. Images of the invading cells were taken with at least three individual fields per chamber by a fluorescent inverted microscope (Olympus IX81 Motorized Inverted Microscope, Japan). The chambers were transferred to an empty well, and 200 μL of Extraction Solution was added to each well. After incubating 10 minutes on an orbital shaker, 100 μL of solution was transferred from each well to a 96-well microtiter plate for measurement under OD 560 nm in a plate reader.

siRNA Transfection

In a six well tissue culture plate, 2×10⁵ cells were seeded per well in 2 ml antibiotic-free normal growth medium supplemented with FBS for incubation overnight. For each gene, siRNA1, siRNA2, siRNA3 were mixed with DharmaFECT transfection reagent with serum-free medium and incubated for 5 min at room temperature. The appropriate transfection mix was added to each well with in the 6-well plate containing fresh medium. After 24 h of transfection, the medium was replaced with complete medium for incubation of 48 h, and the cells were used for further analysis.

HIPK4 cDNA Transient Transfection

Cells were plated into 6-well plate and grown for 1-2 days until reaching 70-80% confluence. Transfection of cells with HIPK4 cDNA was performed utilizing FuGENE® HD Transfection reagent. An amount of 2.5 μg of DNA and 7.5 μL of FuGENE® HD reagent were separately pipeted into 140 μL opti-MEM medium; incubate the FuGENE® HD transfection reagent/DNA mixture for 10-15 minutes at room temperature. Then the mixture was applied onto cells for 7 h in cell culture incubator. The transfection was stopped by replacing medium with complete medium. After 48 h, cells were harvest for further analysis.

### Western Blotting

Briefly, desired cells were harvested and lysed. The protein concentration in the lysates was quantified using the Bradford method with bovine serum albumin as standard. An equal amount of protein of 49 μg was separated by electrophoresis on SDS-polyacrylamide gels and transferred to PVDF membranes. After blocking with 5% nonfat milk, the membranes were incubated with the primary antibodies overnight at the following dilution: HIPK4 (1:500) and β-actin (1:20000). Subsequently, the membranes were incubated with appropriate secondary antibodies. The immunoreactive bands were visualized using enhanced chemiluminescence, as recommended by the manufacturer.

### Example 2—Identification of Metastasis-Driving Kinases in Aggressive Metastatic Breast Cancer Cells

Activated protein kinases are critically involved in the growth and progression of breast cancer, and kinase inhibitors have played an increasingly prominent role in treating cancers. However, there is a lack of known metastasis-specific kinases and corresponding targeted therapy against metastatic breast cancer. It is known that the lung is one of the most frequent sites of metastasis found in metastatic breast cancer patients, and this is a significant contributing factor to breast cancer-associated mortality. Therefore, the lungs were chosen as a primary target to search for targetable kinases that promote metastasis, as well as other organs.

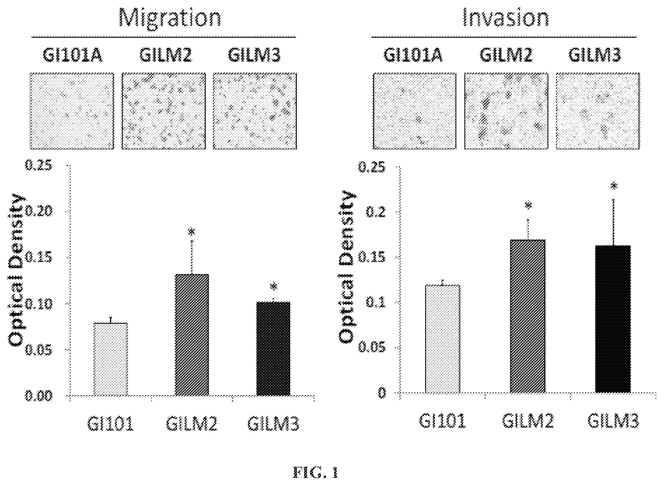

GI101A and its derived GLIM2 and GILM3 clones are highly metastatic human breast cancer cell lines that uniquely develop spontaneous lung metastasis approximately 45 days following xenograft into mouse mammary glands, characteristically representing human breast cancer progression to metastatic disease. GILM2 and GILM3 cells possess enhanced cellular motility and develop significantly more lung metastasis (FIG. 1). Sharing a common genetic background, these cells provide a unique platform for discovering driver kinases and associated substrates responsible for developing metastasis in the lung and other organs.

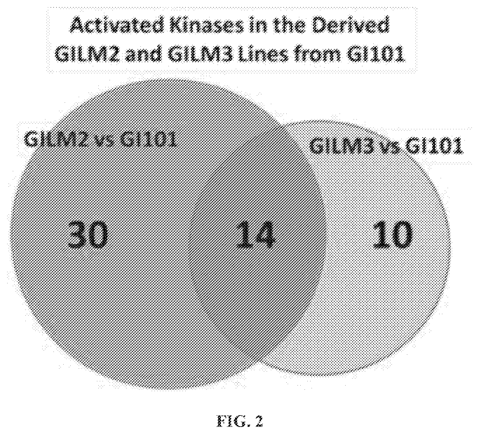

Protein kinase assays were performed using the Full Moon Kinase Antibody Array (Full Moon BioSystems, Sunnyvale, Calif.). These assays were performed for GI101A, GILM2, and GILM3 clones in order to discover kinases that were significantly up- or down-regulated. A set of 14 upregulated kinases was shared between each of the lines (FIG. 2). These kinases are HIPK4, BMPR2, CSNK2A2, LIMK2, PLK1, MAPK10, NTRK3, HERC4, PLK1, RPS6KB2, EPHX1, MYO9B, MAP3K15, and ACTR1B. The screen further indicated that the Homeodomain Interacting Protein Kinase 4 protein was the most significantly upregulated kinase of those identified in the screen. It is speculated that these kinases are associated with the observed metastatic phenotype of GI101A, GILM2, and GILM3 cells.

The homeodomain Interacting Protein Kinases (HIPKs) are a family of 4 (HIPK1-4) highly conserved serine/threonine kinases and nuclear proteins. The first 3 members of the family, HIPK1-3, phosphorylate proteins in response to UV, radiation, cytotoxic stress, or hypoxia, however the identification of roles for HIPK4 has remained elusive. It has previously been reported that HIPK4 phosphorylates p53 at serine 9 both in vitro and in vivo (Arai et al., 2007). Intriguingly, overexpression of HIPK4 in lung cancer cells reduces promoter activity of survivin, resulting in enhanced apoptotic signaling without affecting the promoter activity of Bax, the functional opponent of survivin. Characterization of the HIPK4 protein has revealed that at 616 amino acids in length, it is approximately half the size of the other members of its family, and that they share homology in a conserved N-terminal region containing the serine/threonine kinase catalytic domains (He et al., 2010). Additionally, HIPK4 possesses multiple serine/threonine- and tyrosine-phosphorylation sites, as well as potential sumoylation sites, suggesting a diverse functional profile for HIPK4 in phosphorylating proteins such as p53. No putative role for HIPK4 in regulating cancer cell mobility and metastasis has previously been reported.

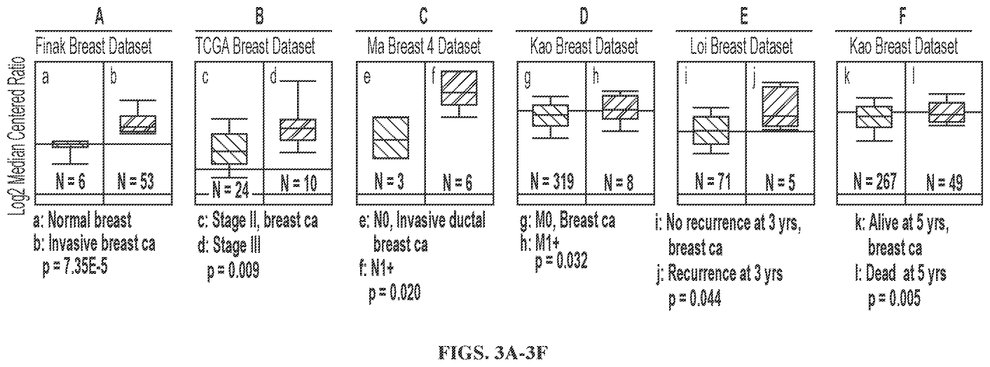

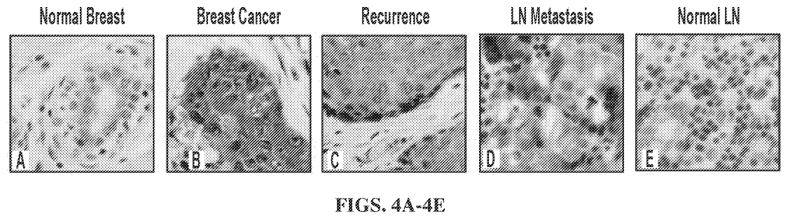

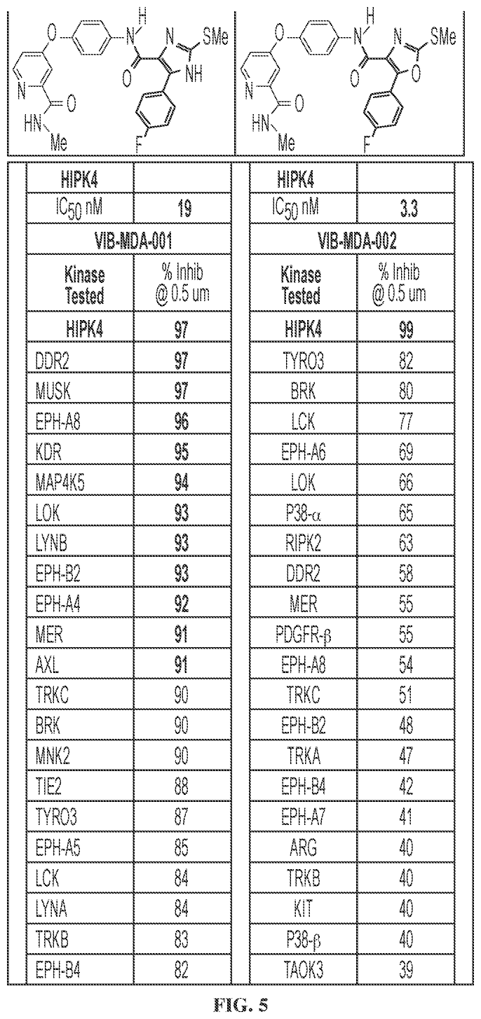

The Oncomine™ gene expression databases were analyzed to understand HIPK4 transcription levels in cancers. Examining breast cancer data sets, it was found that HIPK4 transcription is upregulated in invasive breast cancers and positively correlated with disease stages, lymph node status, metastatic events, recurrence and prognosis (FIG. 3). It was also found that the protein level of HIPK4 was increased in invasive breast cancer tissues, recurrent lesions sites, and lymph nodes with metastatic lesions (FIG. 4). These clinicopathological correlations suggest that HIPK4 plays a key role in promoting metastasis in patients with metastatic breast cancer.

### Example 3—HIPK4 Expression Affects Cellular Migration and Invasion

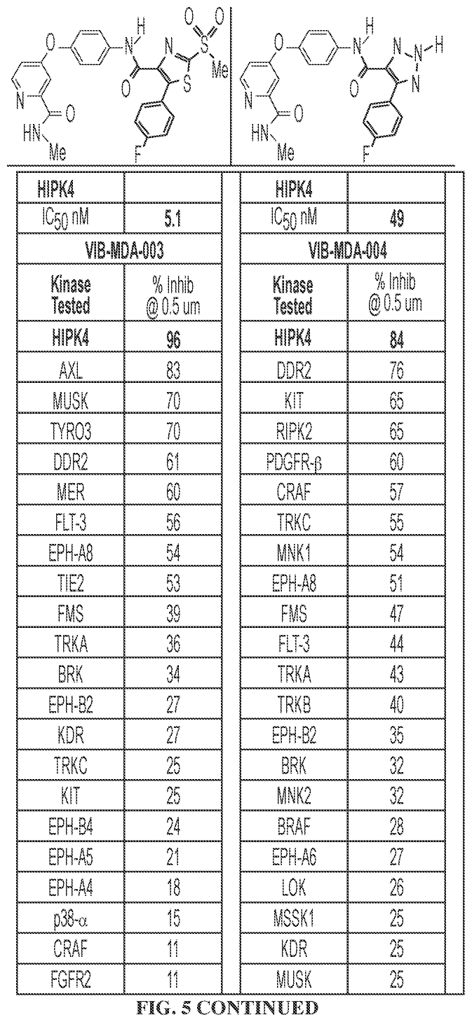

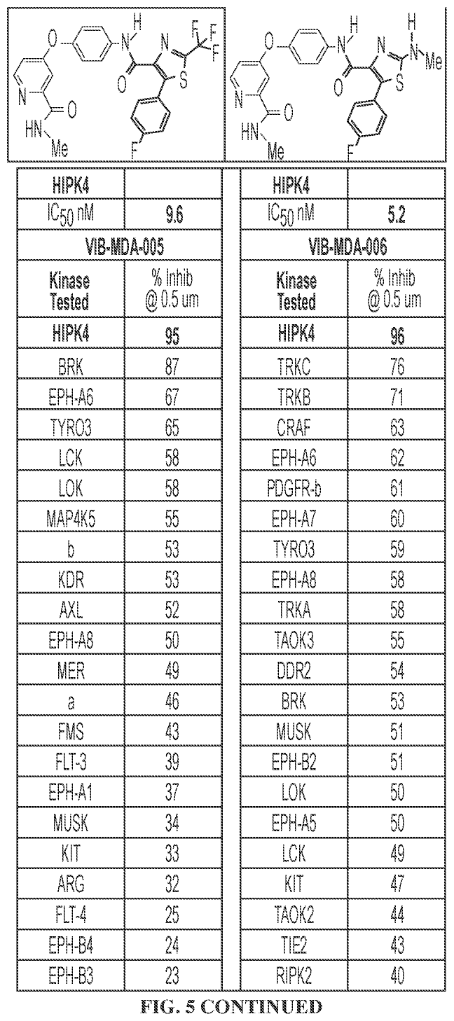

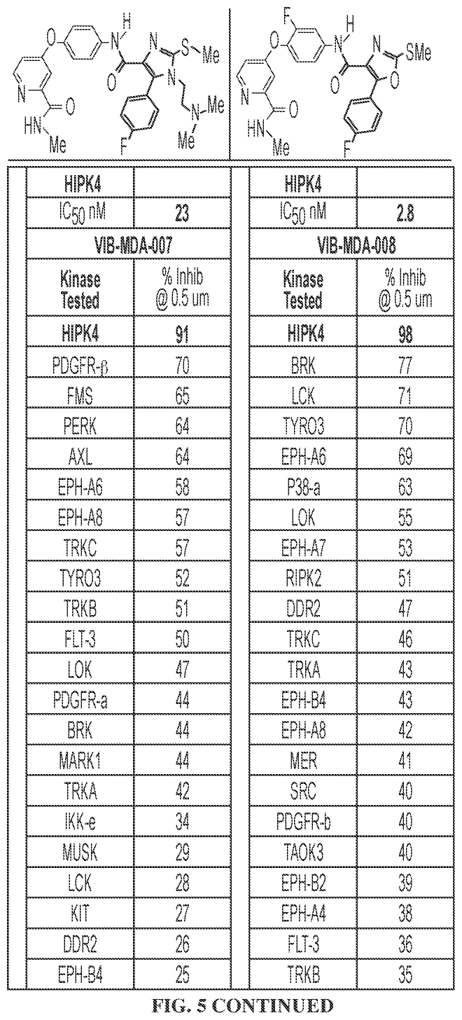

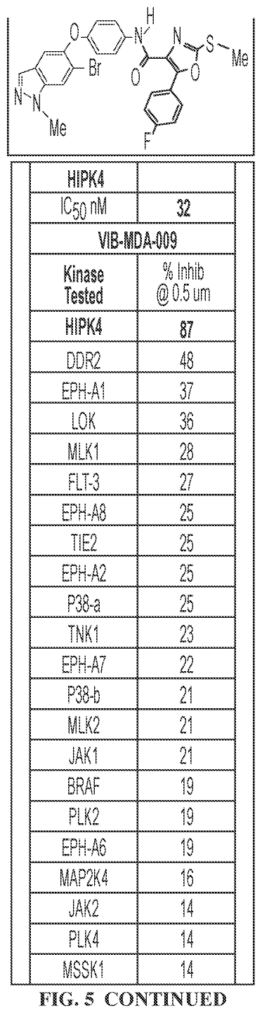

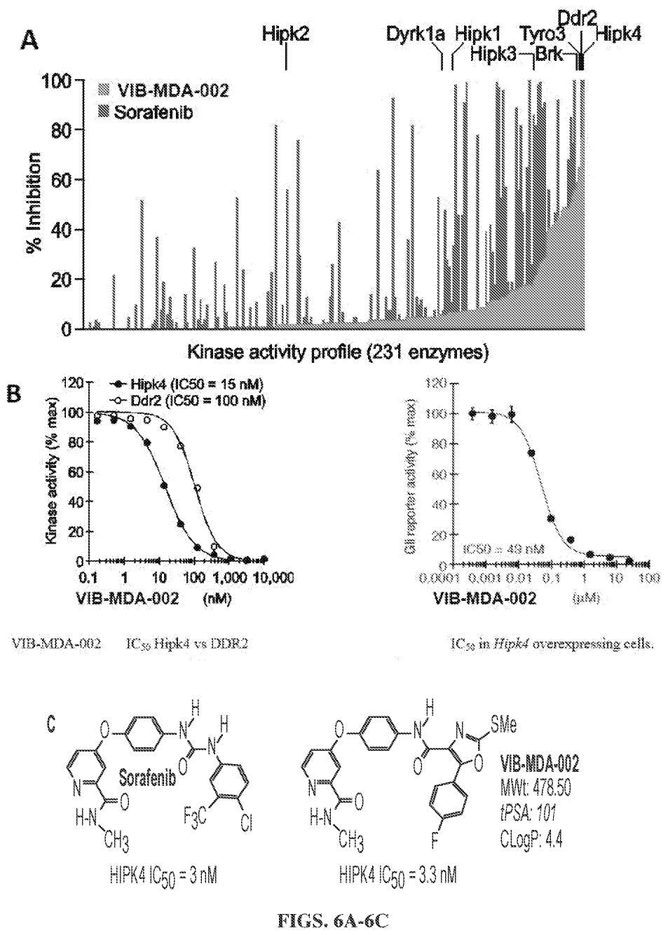

A number of HIPK4 inhibitors have recently been discovered, see for example U.S. Pat. Nos. 9,221,805; 9,833,455 or International PCT Publication WO2013022766, each of which is incorporated herein by reference. These inhibitors have the structures shown in Table 1. Three of these inhibitors, termed VIB-MDA-001, VIB-MDA-002, and VIB-MDA-003, are extremely potent and highly specific for HIPK4 (Table 1, FIG. 5). The inhibition profile of the most highly selective of these is shown in FIG. 6A, in comparison with the FDA approved Sorafenib. This profile shows greatly increased specificity of VIB-MDA-002 for HIPK4 compared to Sorafenib. FIG. 6B more clearly illustrates the selectivity of VIB-MDA-002 for HIPK4, indicating that it has an IC₅₀ of 15 nM., as opposed to the reaction with the second most inhibited kinase, DDR2, in which the IC₅₀ is 100 nM. VIB-MDA-002 has also exhibited excellent transport into cells. The high selectivity of VIB-MD)A-002 in combination with its potency and transportability provide a unique opportunity to study the role HLPK4 plays in cancer metastasis and establish VIB-MDA-002 as a potent small molecule inhibitor for treating metastatic cancers. As shown in FIG. 5, each of the VIB-MDA compounds also inhibit DDR2 (Tyro10) and the TAM kinase family (Including Tyro3, Axl (Tyro7), and Mer (Tyro12)), with VIB-MD)A-002 exhibiting the highest potency for HIPK4, DDR2, Tyro3, and the Breast Tumor Kinase (BRK). DDR kinases, TAM kinases, and BRK kinases are critically involved in regulating cellular migration, the epithelial-mesenchymal transition (EMT) and metastasis in many cancers, and may thus serve as potential HIPK4 downstream substrates.

TABLE 1
HIPK4 inhibitors

| | |
|---|---|
| VIB- MDA- 001 | 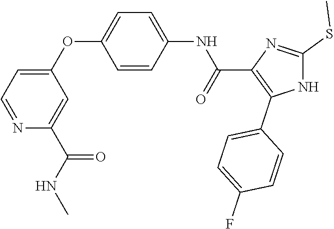 [SMILES: CNC(=O)c1cc(Oc2ccc(NC(=O)c3nc(SC)[nH]c3-c3ccc(F)cc3)cc2)ccn1] |
| VIB- MDA- 002 | 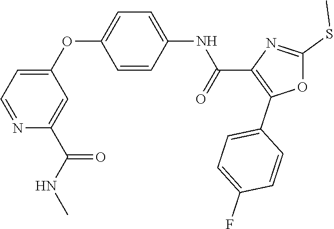 [SMILES: CNC(=O)c1cc(Oc2ccc(NC(=O)c3nc(SC)oc3-c3ccc(F)cc3)cc2)ccn1] |
| VIB- MDA- 003 | 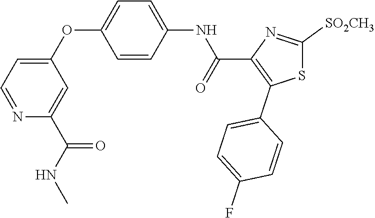 [SMILES: CNC(=O)c1cc(Oc2ccc(NC(=O)c3nc(S(C)(=O)=O)sc3-c3ccc(F)cc3)cc2)ccn1] |
| VIB- MDA- 004 | 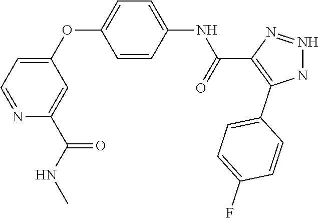 [SMILES: CNC(=O)c1cc(Oc2ccc(NC(=O)c3nn[nH]c3-c3ccc(F)cc3)cc2)ccn1] |
| VIB- MDA- 005 | 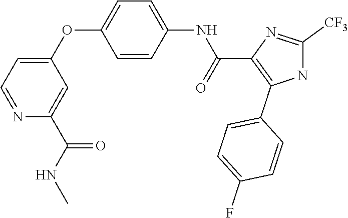 [SMILES: CNC(=O)c1cc(Oc2ccc(NC(=O)c3nc(C(F)(F)F)[nH]c3-c3ccc(F)cc3)cc2)ccn1] |
| VIB- MDA- 006 | 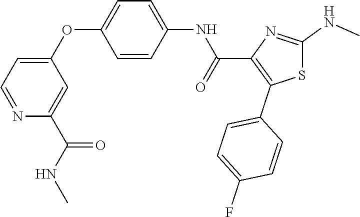 [SMILES: CNC(=O)c1cc(Oc2ccc(NC(=O)c3nc(NC)sc3-c3ccc(F)cc3)cc2)ccn1] |
| VIB- MDA- 007 | 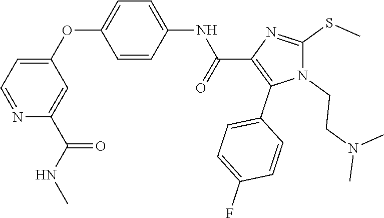 [SMILES: CNC(=O)c1cc(Oc2ccc(NC(=O)c3nc(SC)n(CCN(C)C)c3-c3ccc(F)cc3)cc2)ccn1] |
| VIB- MDA- 008 | 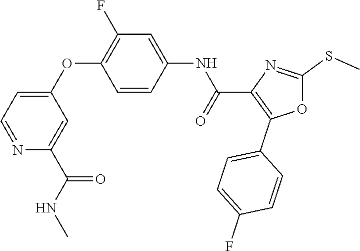 [SMILES: CNC(=O)c1cc(Oc2ccc(NC(=O)c3nc(SC)oc3-c3ccc(F)cc3)cc2F)ccn1] |
| VIB- MDA- 009 | 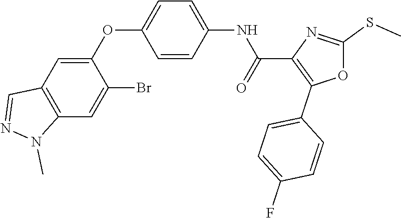 [SMILES: CSc1nc(C(=O)Nc2ccc(Oc3cc4cnn(C)c4cc3Br)cc2)c(-c2ccc(F)cc2)o1] |

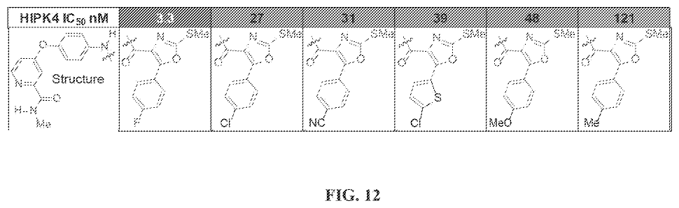

Variation of the aryl scaffold modulates potency (FIG. 12).

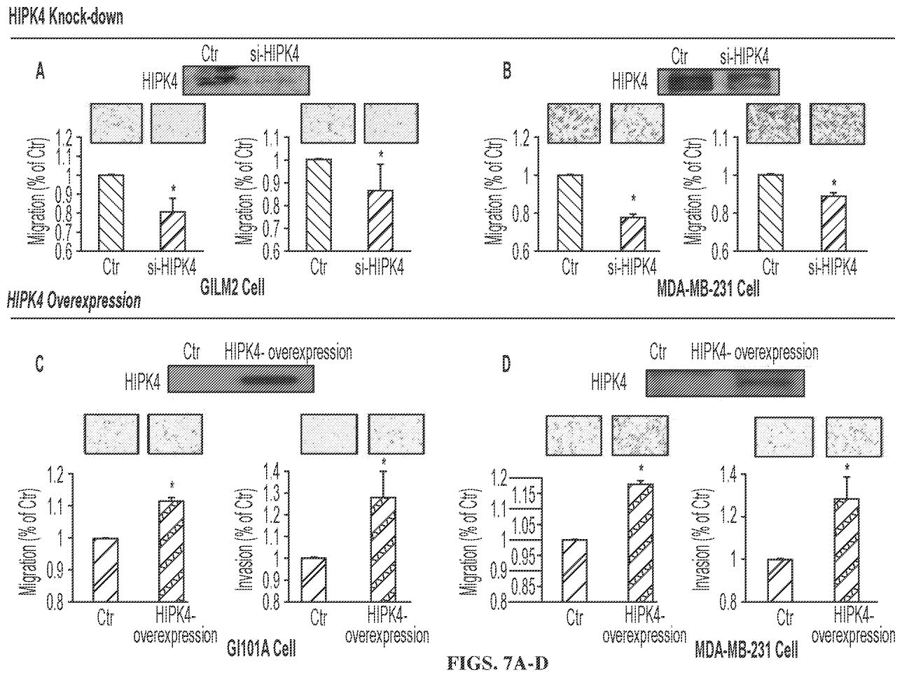

In preliminary studies, it has been shown that loss of HIPK4 via transient siRNA knockdown significantly reduced migration and invasion in five highly metastatic breast cancer cell lines (triple negative breast cancer cell lines: MDA-MB231, MDA-MB468; highly metastatic ER-positive breast cancer lines GI101A, GILM2, and GILM3) and 1 pancreatic cancer cell line (Panc-1) (FIGS. 7A-7B). Consistent with this finding, gain of HIPK4 by transient overexpression enhanced breast cancer cell migration and invasion (FIGS. 7C-7D). These results show that altered expression of HIPK4 significantly impacts the motility of metastatic cancer cells.

### Example 4—Modulation of HIPK4 Activity Affects Metastasis

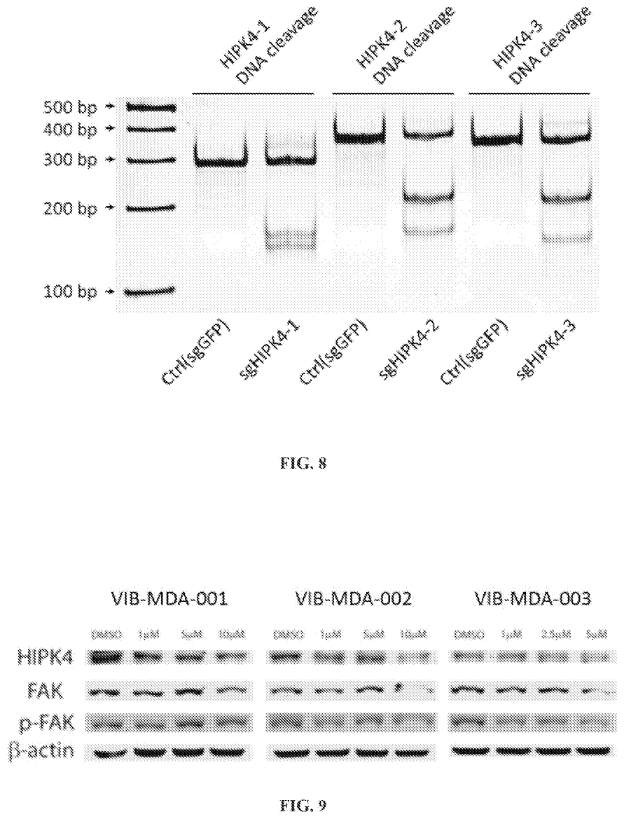

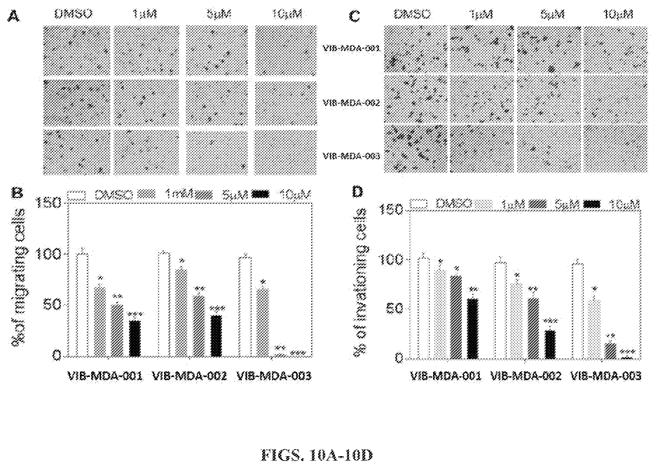

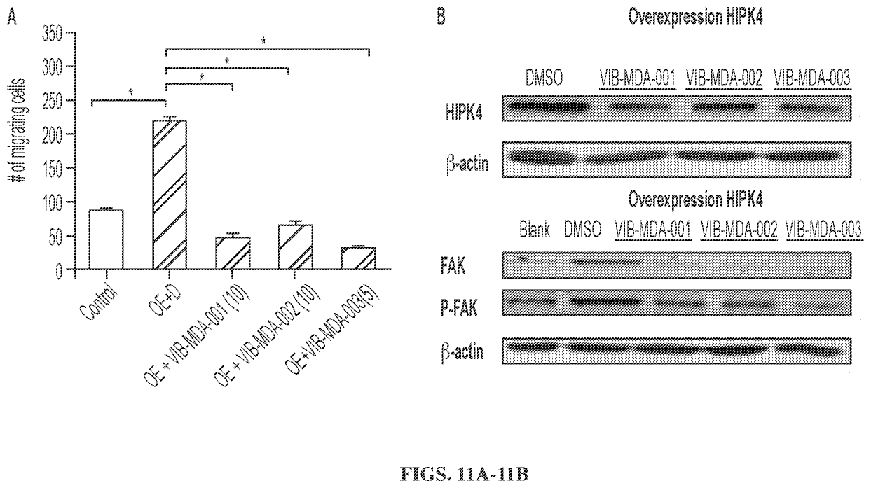

Given the results presented herein, it is expected that modulation of HIPK4 activity will affect the development of other metastases. To examine this in lung cancers, loss of HIPK4 cell lines have been constructed using modified Edit-R CRISPR-Cas9 gene engineering system (Dharmacon) was used with inducible HIPK-CRISPR-KO vectors and stable clones in GI1101A and derivative cell lines to generate specific, high-efficiency, permanent knockouts of HIPK4 gene function (FIG. 8). These knockouts will be compared to otherwise identical, unmodified cell lines with or without HIPK4 inhibitor treatment. Selective inhibition of HIPK4 expression is shown for VIB-MDA-001, VIB-MDA-002, and VIB-MDA-003 in FIG. 9. To determine the effect of these inhibitors on cell migration and invasion, each cell line was treated with either DMSO, or 1, 5, or 10 μM of each of the HIPK4 inhibitors. The results for GILM3 are shown in FIGS. 10A and 10B. These results demonstrate the potential for efficient in vivo inhibition and the possibility for clinical translation.

Gain of HIPK4 function clones have been selected for stable, inducible pUHD-Tet-on/off HIPK4 function. HIPK4 was overexpressed in MDA-MB-231 cells and it was found that HIPK4 expression stimulates cellular migration (FIG. 11A), while VIB-MDA-001/-002/-003 compounds significantly inhibit the migration from HIPK-overexpression and expression of HIPK and FAK/pFAK in triple negative breast cancer cells (FIG. 11B).

Despite major advances in detecting and treating breast cancer over the last 20 years, the majority of deaths from breast cancer are a direct result of metastasis developed in the lung or other organs. However, current understanding of key determinants in developing metastasis is limited, accompanied by the lack of targeted strategies for suppressing existing metastasis or blocking the development of new metastases. Thus, characterization of HIPK4's previously undefined role in promoting metastasis represents discovery of a key determinant highly relevant to the aggressive phenotype of metastatic breast cancer. Further identification of key determinants of metastasis through the study of HIPK4 and its substrates will reduce the morality associated with metastatic breast cancer by developing and evaluating novel selective HIPK4 inhibitors for suppressing metastasis in vivo.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Al-Beiti and Lu, The EMBO Journal, 25:3955-3965, 2006.
Arai et al., FEBS Letters, 581:5649-5657, 2007.
Blaquiere and Verheyen, Curr Top Dev Biol., 123:73-103, 2017.
Cao et al., Lab Invest., 91(4): 598-608, 2011.
Chalazonitis et al., J. Neuroscience, 31:13746-13757, 2011.
Chen and Verheyen, Current Biology, 22:1582-1586, 2012.
Cheng et al., Molecular Medicine Reports, 5:1251-1255, 2012.
Cozza et al., PLoS ONE, 9:e89176, 2014.
Davis et al., Nat. Biotechnol., 29:1046-1051, 2011.
de la Vega et al., Biochemica et Biophysica Acta, 1813:283-297, 2011.
Deshmukh et al., Oncogene, 27:4745-4751, 2008.
D'orazi et al., Clinical Cancer Research, 12:735-741, 2006.
Ecsedy et al., Mol Cell Biol., 23(30):950-960, 2003.
He et al., Molecular and Cellular Pharmacology, 2:61-68, 2010.
Hofmann et al., Cancer Res., 63: 8271-8277, 2003.
Hikasa and Sokol, J. Biol. Chem., 286:12093-12100, 2011.
Hikasa et al., Developmental Cell, 19:521-532, 2010.
Iacovelli et al., Cell Prohferation., 42:373-384, 2009.
Jacob et al., British Journal of Cancer, 101:722-733, 2009.
Kim et al., J. Biol Chem., 273(40): 25875-9, 1998.
Kondo et al., Proc. Natl. Acad. Sci. USA, 100:5431-5436, 2003.
Lavra et al., PLoS ONE, 6:e20665, 2011.
Lee et al., Dev. Biol., 325:263-272, 2009.
Moilanen et al., Mol. Biol. Cell, 9(9):2527-43, 1998.
Neklesa et al., Pharmacol. Ther., 174:138-144, 2017.
Pierantoni et al., Biochem. And Biophys. Res. Comm., 290:942-947, 2002.
Poon et al., Curr. Biol., 22:1587-1594, 2012.
Rey et al., Cell Cycle., 12:1879-1891, 2013.
Ricci et al., J. Cell Phys., 228:235-241, 2013.
Rinaldo et al., Biochem Biophysica Acta, 1783(11):2124-9, 2008.
Ritter et al., ACS Cent. Sci., 3(9):944-948, 2017.
Rochat-Steiner et al., J Exp Med., 192(8):1165-74, 2000.
Saul and Schmitz, J. Mol Med, 91:1051-1058, 2013.
Shigeki et al., FEBS let., 581:5649-5657, 2007.
Sjolund et al., Proc. Natl. Acad. Sci. USA., 111:7373-7378, 2014.
Tan et al., J. Cell Biochem., 115:1762-1767, 2014.
Trapasso et al., DNA Cell Biol., 28:161-1674, 2009.
U.S. Pat. No. 9,221,805
U.S. Pat. No. 9,833,455
Van der Laden et al., Cell Comm. Sig., 13:3, 2015.
Wei et al., Proc. Natl. Acad. Sci. USA, 104:13040-13045, 2007.
WO2004087901
WO2013022766
Yu et al., Neurology, 73:1526-1531, 2009.
Zhang et al., Proc Natl Acad Sci USA., 102(8):2802-7, 2005.

What is claimed is:

1. A method of treating cancer in a subject comprising administering an effective amount of an inhibitor of homeodomain interacting protein kinase 4 (HIPK4) to the subject.

2. The method of claim 1, further defined as a method for preventing and inhibiting cancer metastasis.

3. The method of claim 1, further defined as a method for inhibiting cancer cell migration or invasion.

4. The method of claim 1, wherein the cancer is an invasive or progressive cancer.

5. The method of claim 1, wherein the cancer is oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer.

6. The method of claim 5, wherein the cancer is a breast cancer.

7. The method of claim 6, wherein the cancer is an invasive breast cancer with or without metastatic diseases or lesions.

8. The method of claim 1, wherein the subject has a metastatic cancer.

9. The method of claim 8, wherein the subject has a metastasis developed in the lungs, brain, bone, or liver.

10. The method of claim 8, wherein the subject has a metastasis in multiple organs.

11. The method of claim 1, wherein the inhibitor of HIPK4 is an inhibitory nucleic acid molecule.

12. The method of claim 11, wherein the inhibitory nucleic acid is a siRNA, shRNA, miRNA, dsRNA, a ribozyme or antisense nucleic acid.

13. The method of claim 1, wherein the inhibitor of HIPK4 is a small molecule kinase inhibitor.

14. The method of claim 13, wherein the small molecule inhibitor is a compound of the formula: 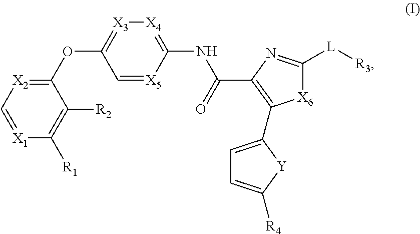
wherein:
X₁, X₂, X₃, X₄, and X₅ are each independently —N═ or —C(Rₓ)═, wherein:
Rₓ is —H, —F, —Cl, —Br, or —CH₃,
X₆ is —O—, —S—, —NH—, —N(CH₃)—, or —N[(CH₂)ₘN(CH₃)₂]—, wherein:
m is 2, 3, or 4;
R₁ and R₂ are each independently hydrogen, —NH₂, —Cl, —C(O)NHCH₃, —NHC(O)CH₃, —NHC(O)-cyclopropyl, —NHCO₂CH₃; or
R₁ and R₂ are taken together and form a cycloalkene_{(C≤12)}, a heterocycloalkene_{(C≤12)}, an arene_{(C≤12)}, a heteroarene_{(C≤12)}, or a substituted version of any of these groups;
R₃ is —H, —F, —CH₃, —CH₂CH₃, cyclopropyl, —C(O)CH₃, or —(CH₂)ₙN(CH₃)₂,
wherein:
n is 2, 3, or 4;
R₄ is —H, —F, —Cl, —CN, —OH, —CH₃, or —OCH₃;
L is a covalent bond, —CH₂—, —CF₂—, —CH(OH)—, —O—, —S—, —S(O)—, —S(O)₂—, —NH—, or —CH₂O—; and
Y is —CH═CH—, —CF═CH—, or —S—.

15. The method of claim 13, wherein the small molecule inhibitor is VIB-MDA-001, VIB-MDA-002, VIB-MDA-003, VIB-MDA-004, VIB-MDA-005, VIB-MDA-006, VIB-MDA-007, VIB-MDA-008, or VIB-MDA-009.

16. The method of claim 15, wherein the small molecule inhibitor is VIB-MDA-001, VIB-MDA-002, or VIB-MDA-003.

17. The method of claim 16, wherein the small molecule inhibitor is VIB-MDA-001.

18. The method of claim 16, wherein the small molecule inhibitor is VIB-MDA-002.

19. The method of claim 16, wherein the small molecule inhibitor is VIB-MDA-003.

20. The method of claim 14, wherein the small molecule inhibitor comprises ¹⁸F.

21. The method of claim 14, wherein R₄ is ¹⁸F.

22. The method of claim 1, wherein the subject has been determined to have a cancer with an elevated level of HIPK4 expression.

23. The method of claim 1, wherein the inhibitor of HIPK4 is administered more than once.

24. The method of claim 23, wherein the inhibitor of HIPK4 is administered 1, 2, 3, 4, 5, 6, or more times per week.

25. The method of claim 1, wherein the inhibitor of HIPK4 is administered daily.

26. The method of claim 25, wherein the inhibitor of HIPK4 is administered on a continuous basis.

27. The method of claim 1, further comprising administering an additional anti-cancer therapy.

28. The method of claim 27, wherein the additional anti-cancer therapy is chemotherapy, radiotherapy, gene therapy, surgery, hormonal therapy, anti-angiogenic therapy or immunotherapy.

29. The method of claim 1, wherein the inhibitor of HIPK4 is administered intravenously, subcutaneously, intraosseously, orally, transdermally, via inhalation, in sustained release, in controlled release, in delayed release, as a suppository, or sublingually.

30. The method of claim 1, wherein administering the inhibitor of HIPK4 comprises local, regional or systemic administration.

31. The method of claim 1, wherein the subject is a human.

32. A method of treating a subject having a cancer comprising:
(a) obtaining a sample from the subject,
(b) determining the level of homeodomain interacting protein kinase 4 (HIPK4) expression or kinase function in the sample; and
(c) administering an effective amount of an inhibitor of HIPK4 to a subject determined to have an elevated level of HIPK4 expression or kinase function.

33. A method of predicting a response to an inhibitor of HIPK4 in a subject having a cancer comprising detecting the level of HIPK4 expression or kinase function in a tissue sample obtained from the subject, wherein if the sample exhibits increased expression or kinase activity of HIPK4, then the subject is predicted to have a favorable response to a HIPK4 inhibitor therapy.

34. The method of claim 32 or 33, wherein the level of HIPK4 expression is the level of HIPK4 mRNA expression.

35. The method of claim 32 or 33, wherein the level of HIPK4 expression is the level of HIPK4 protein expression.

36. The method of claim 32 or 33, wherein the level of HIPK4 activity is the level of HIPK4 kinase function.

37. The method of claim 32 or 33, wherein the sample is from saliva, blood, urine, or tumor tissue.

38. The method of claim 34, wherein the level of HIPK4 expression is determined by PCR analyses.

39. The method of claim 35, wherein the level of HIPK4 function is determined by kinase analyses.
